(19) United States
(12) Patent Application Publication
(10) Pub. No.: US 2021/0259680 A1
(43) Pub. Date: Aug. 26, 2021

(54) SURGICAL GRASPER WITH INTEGRATED SUTURE PULLEY

(71) Applicant: Activ Surgical, Inc., Boston, MA (US)

(72) Inventors: Liam O'Shea, Westwood, MA (US); Thomas J. Calef, Boston, MA (US); Dana Zitnick, Warwick, RI (US)

(21) Appl. No.: 17/191,200

(22) Filed: Mar. 3, 2021

Related U.S. Application Data
(63) Continuation of application No. PCT/US2019/050308, filed on Sep. 10, 2019.
(60) Provisional application No. 62/729,069, filed on Sep. 10, 2018.

(51) Int. Cl. A61B 17/04; A61B 17/28; A61B 34/30

(52) U.S. Cl. CPC A61B 17/0491; A61B 17/2816; A61B 2017/0608; A61B 34/30; A61B 17/282

(57) ABSTRACT

A surgical grasper with an integrated suture pulley engages a suture during a suturing process. The grasper includes a jaw member pivotally connected to another jaw member, each having a suture engagement surface facing the suture engagement surface of the other. One jaw member includes a suture pivot post extending from its suture engagement surface. The other jaw member has an opening in its suture engagement surface sized and arranged to receive the pivot post. The grasper can be operated to position the jaw members in a suture grasping mode, in which the pivot post is received in the opening and the suture engagement surfaces securely engage the suture to inhibit movement of the suture. The jaw members can also be positioned in a pulley mode, in which the grasper slidably supports the suture and the pivot post acts as a pivot point and bearing surface for the suture.

### CROSS REFERENCE TO RELATED APPLICATION

This application is a continuation application of International Application No. PCT/US2019/050308, filed on Sep. 10, 2019, which application claims priority from U.S. Provisional Patent Application No. 62/729,069, filed on Sep. 10, 2018, which applications are incorporated herein by reference in their entirety for all purposes.

### BACKGROUND

The present application relates generally to surgical devices for suturing body tissue and, more particularly, to devices for handling suture material during the suturing process.

Suturing is used to close the edges of a wound or incision and to repair damaged tissue during surgery. Sutures can be applied using a suturing device having a needle, which can be manipulated to draw a suture through the tissue at the edges of the incision. The suture can be tightened to draw the edges of the tissue together, and then knotted and the suture tail trimmed.

When suturing, particularly in a minimally invasive surgery (MIS), there is a need to control the suture material so that it stays out of the way of the suture path and also so that the entire length of the suture can be used. This would avoid the need to pass suture/needles in and out when there is extensive knot tying in a procedure. In current MIS procedures, the surgeon typically clears the area prior to tying a knot with one of their available hand tools or grasps the suture and pulls it out of the way. However, when grasping the suture, if it is too close to the exit point of the tissue, forces can be exerted on the tissue that can result in poor patient outcomes (tissue trauma, poor anastomosis results, etc.) or inefficient use of time in the operating room (suture breakage, etc.).

### BRIEF SUMMARY

In accordance with one or more embodiments, a surgical grasper with an integrated suture pulley for engaging a suture during a suturing process is disclosed. The grasper includes a jaw member pivotally connected to another jaw member, each having a suture engagement surface facing the suture engagement surface of the other. One of the jaw members includes a suture pivot post extending from its suture engagement surface, and the other jaw member has an opening in its suture engagement surface sized and arranged to receive the suture pivot post. The grasper can be operated to position the jaw members relative to each other in a suture grasping mode, in which the suture pivot post is received in the opening and the suture engagement surfaces securely engage the suture to inhibit movement of the suture relative to the grasper. The jaw members can also be positioned relative to each other in a pulley mode, in which the grasper slidably supports the suture and the suture pivot post acts as a pivot point and bearing surface for the suture.

In accordance with one or more further embodiments, a method is disclosed for handling a suture using surgical grasper having opposing jaws during a suturing process. The method includes (a) positioning the jaws of the surgical grasper on opposite sides of the suture; (b) moving the jaws relative to each other to a grasping mode in which the grasper fixedly holds a portion of the suture and inhibits movement of the suture relative to the grasper for one part of the suturing process; and (c) moving the jaws relative to each other to a pulley mode in which the grasper slidably supports the suture at a pivot point and permits the suture to slide relative to the grasper for another part of the suturing process.

In accordance with one or more further embodiments, a surgical grasper for engaging a suture in multiple modes of operation is disclosed. The grasper includes a first jaw member having a suture engagement surface and a suture pivot post extending from the suture engagement surface at a distal end of the first jaw member. The grasper also includes a second jaw member opposing the first jaw member. The second jaw member has a suture engagement surface facing the suture engagement surface of the first jaw member. The second jaw member also has an opening in the suture engagement surface at a distal end of the second jaw member configured to receive the suture pivot post. The first jaw member and said second jaw member are pivotally connected at a proximal end of each jaw member. One or both of the first jaw member and the second jaw member can be actuated to move relative to the other among at least a first grasper position, a second grasper position, and a third grasper position. In the first grasper position, the distal ends of the first jaw member and the second jaw member are separated such that a gap is present between the suture pivot post in the first jaw member and the opening in the second jaw member such that the suture can be inserted in the grasper. In the second position, the distal ends of the first and second jaw members are moved closer together than in the first position and the post engages the opening but the suture engagement surfaces are sufficiently separated to enable the suture to slidably move using the post as a pivot point and bearing surface. In the third position, the distal ends of the first and second jaw members are moved closer together than in the second position and the suture engagement surfaces engage the suture to inhibit movement of the suture relative to the grasper.

In accordance with one or more further embodiments, a robotic suturing system is disclosed. The system includes a first robotic arm, a second robotic arm, a suturing device operatively coupled to the first robotic arm, a suture handling device operatively coupled to the second robotic arm, and a control system. The control system is operatively coupled to the first robotic arm, the second robotic arm, the suturing device, and the suture handling device to control operation thereof to cooperatively perform a suturing procedure on a patient. The suture handling device includes a surgical grasper for engaging a suture. The grasper includes a first jaw member having a suture engagement surface and a suture pivot post extending from the suture engagement surface at a distal end of the first jaw member. The grasper also includes a second jaw member opposing the first jaw member. The second jaw member has a suture engagement surface facing the suture engagement surface of the first jaw member. The second jaw member also has an opening in the suture engagement surface at a distal end of the second jaw member configured to receive the suture pivot post. The first jaw member and said second jaw member are pivotally connected at a proximal end of each jaw member. One or both of the first jaw member and the second jaw member can be actuated to move relative to the other among at least a first grasper position, a second grasper position, and a third grasper position. In the first grasper position, the distal ends of the first jaw member and the second jaw member are separated such that a gap is present between the suture pivot post in the first jaw member and the opening in the opening in the second jaw member such that the suture can be inserted in the grasper. In the second position, the distal ends of the first and second jaw members are moved closer together than in the first position and the post engages the opening but the suture engagement surfaces are sufficiently separated to enable the suture to slidably move using the post as a pivot point and bearing surface. In the third position, the distal ends of the first and second jaw members are moved closer together than in the second position and the suture engagement surfaces engage the suture to inhibit movement of the suture relative to the grasper.

### BRIEF DESCRIPTION OF THE DRAWINGS

FIG. 1 is a simplified block diagram illustrating an exemplary robotic suturing system in accordance with one or more embodiments.

FIGS. 2 and 3 illustrate operation of the suturing system in a suturing procedure.

FIG. 4 illustrate an exemplary surgical grasper in the suturing system in accordance with one or more embodiments.

FIGS. 5A-5C illustrate various operating modes of the surgical grasper in accordance with one or more embodiments.

### DETAILED DESCRIPTION

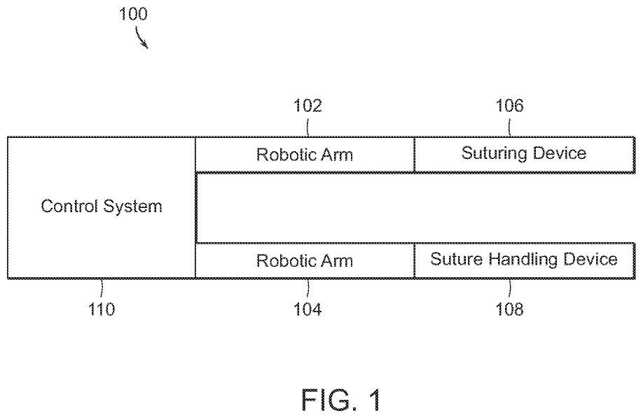

FIG. 1 is a block diagram schematically illustrating one example of a robotic suturing system 100 in accordance with one or more embodiments. The robotic suturing system can be used to perform a suturing procedure on a patient lying on an operating table during surgery.

The system includes a first robotic arm 102 operatively coupled to a suturing device 106. The system also includes a second robotic arm 104 operatively coupled to a suture handling device 108. Each robot arm is connected to a computer control system 110, which controls movement of the robot arms and operation of the suturing device 106 and the suture handling device 108.

Examples of robotic arms that can be configured for use in the suturing system include those available from KUKA Robotics, Medineering Surgical Robotics, and ABB Robotics, among others.

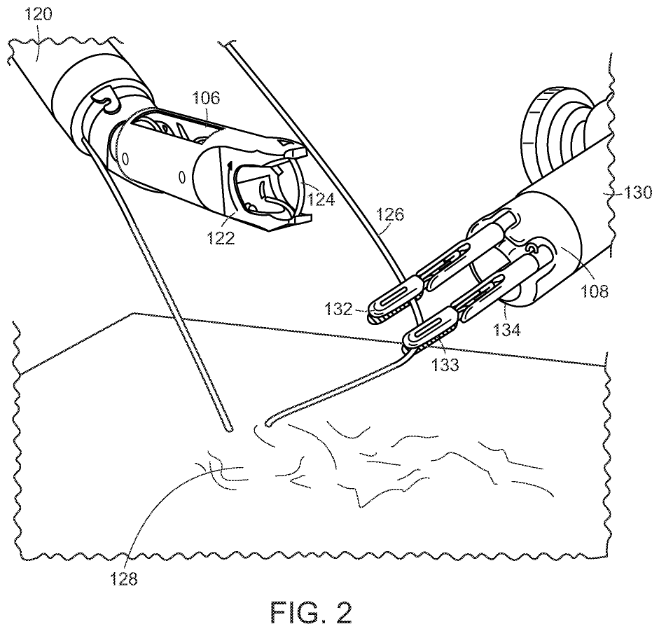

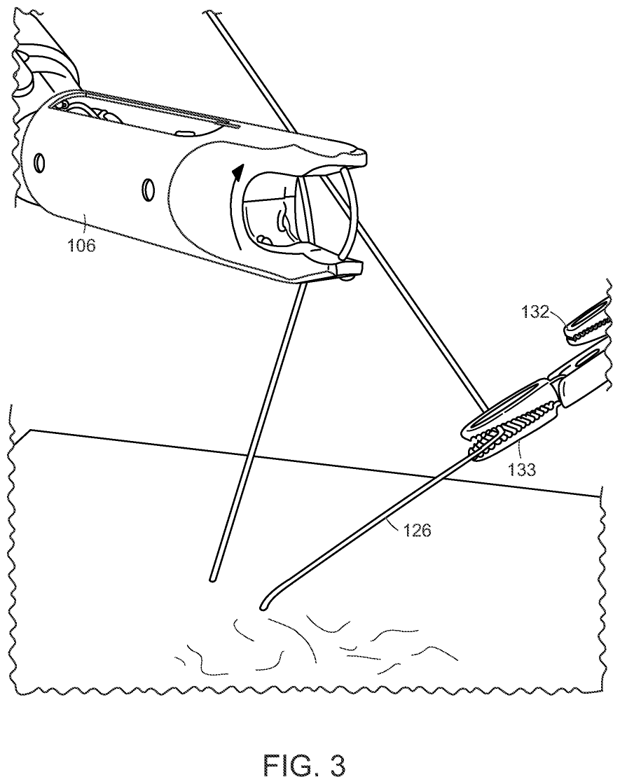

FIGS. 2 and 3 illustrate operation of the suturing device 106 and the suture handling device 108 in a suturing procedure. The suturing device 106 includes a tubular housing 120 having a proximal end (not shown) coupled to the first robotic arm 102 and an opposite distal end having a suturing end effector 122. The suturing end effector 122 includes a curved needle 124 that can be manipulated to draw a suture 126 through tissue 128. The suture handling device 108 works cooperatively with the suturing device 106 to place and knot the suture 126 in the tissue 128 as shown in FIGS. 2 and 3.

The suture handling device 108 includes an elongated tubular housing 130 having a proximal end (not shown) connected to the second robotic arm 104 and an opposite distal end having first and second suture grasping end effectors 132, 133 and a laparoscope 134.

The elongated tubular housing 130 includes three internal working channels extending longitudinally along the length of the tubular housing 130. The first and second channels house the first and second suture grasping instruments 132, 133, respectively. The third channel houses the laparoscope 134.

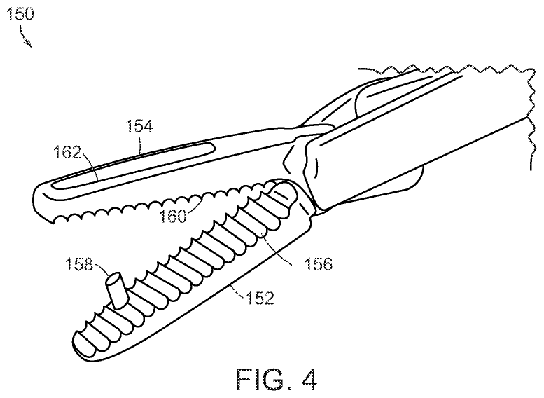

The first and second suture grasping end effectors 132, 133 each include a surgical grasper 150 shown in FIG. 4. The surgical grasper includes a jaw assembly comprising a first jaw member 152 pivotally connected to a second jaw member 154.

By way of non-limiting example, the jaw assembly can be a 2-3.5 mm surgical grasper.

The first jaw member 152 has a suture engagement surface 156 and a suture pivot post 158 extending from the suture engagement surface 156 at a distal end of the first jaw member 152.

The second jaw member 154 opposes the first jaw member 152. The second jaw member 154 has a suture engagement surface 160 facing the suture engagement surface 156 of the first jaw member 152. The second jaw member 154 also has an opening 162 in its suture engagement surface at a distal end of the second jaw member 154 sized and arranged to receive the suture pivot post 158.

In accordance with one or more embodiments, the suture engagement surfaces 156, 160 of the first and second jaw members 152, 154 have complementary serrations (e.g., V or U-shaped teeth) to more securely grip the suture.

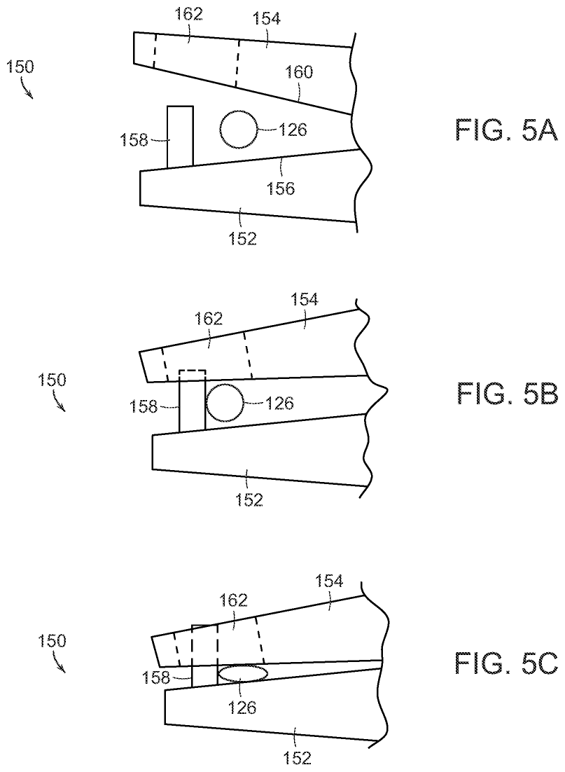

The first and second jaw members 152, 154 are pivotally connected at a proximal end of each jaw member. One or both of the first and second jaw members 152, 154 can be actuated to move relative to the other among at least a first open position, a second suture pulley mode position, and a third suture grasping mode position as illustrated in FIGS. 5A, 5B, and 5C, respectively.

In the first open position (FIG. 5A), the distal ends of the first and second jaw members 152, 154 are separated such that there is a gap between the suture pivot post 158 in the first jaw member 152 and the opening 162 in the second jaw member 154 such that the suture 126 can be inserted in the grasper 150.

In the second suture pulley mode position (FIG. 5B), the distal ends of the first and second jaw members 152, 154 are moved closer together than in the first position. The post 158 engages the opening 162, but the suture engagement surfaces 156, 160 are sufficiently separated to enable the suture 126 to slidably move relative to the grasper 150 using the post 158 as a pivot point and bearing surface.

In the third suture grasping mode position (FIG. 5C), the distal ends of the first and second jaw members 152, 154 are moved closer together than in the second position and the suture engagement surfaces 156, 160 securely grip the suture 126, inhibiting movement of the suture 126 relative to the grasper 150.

FIGS. 2 and 3 illustrate use of the grasping end effectors 132, 133 operating cooperatively with the suture device 106 in a suturing procedure. In FIG. 2, the graspers 132, 133 are operating in the third suture grasping mode, where the suture 126 is securely and fixedly held relative to the graspers. In FIG. 3, the grasper 133 is used in the second suture pulley mode, where the suture 126 slides through the grasper using the post 158 as a pivot point and bearing surface.

The suturing system including the grasper with integrated suture pulley can be utilized in a variety of robotic surgical equipment, including autonomous robotic surgical systems as well as surgeon-controlled robotic equipment.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-14. (canceled)

15. A system for suturing, comprising:
a surgical grasper having a plurality of grasper positions, wherein the surgical grasper comprises a first jaw member pivotally connected to a second jaw member, wherein the first jaw member comprises a suture pivot post extending from a suture engagement surface of the first jaw member, wherein the second jaw member comprises an opening disposed on a suture engagement surface of the second jaw member,
wherein the plurality of grasper positions comprise a suture pulley grasper position in which the suture pivot post of the first jaw member is positioned adjacent to or partially within the opening of the second jaw member such that the suture pivot post acts as a pivot point and bearing surface to slidably support a suture.

16. The system of claim 15, wherein the plurality of grasper positions comprise an open grasper position in which a distal end of the first jaw member and a distal end of the second jaw member are separated to provide a gap between the suture pivot post of the first jaw member and the opening of the second jaw member.

17. The system of claim 16, wherein the gap between the suture pivot post and the opening is sized to permit insertion of the suture into the surgical grasper.

18. The system of claim 15, wherein the plurality of grasper positions comprise a closed grasper position in which the first jaw member and the second jaw member are positioned adjacent to or placed in contact with each other such that the suture engagement surfaces of the first jaw member and the second jaw member securely engage the suture to reduce or inhibit movement of the suture.

19. The system of claim 18, wherein the suture pivot post of the first jaw member is received or positioned within the opening of the second jaw member when the surgical grasper is placed in the closed grasper position.

20. The system of claim 15, wherein the plurality of grasper positions comprise:
(a) an open grasper position in which a distal end of the first jaw member and a distal end of the second jaw member are separated to provide a gap between the suture pivot post of the first jaw member and the opening of the second jaw member;
(b) the suture pulley grasper position; and
(c) a closed grasper position in which the first jaw member and the second jaw member are positioned adjacent to or placed in contact with each other such that the suture engagement surfaces of the first jaw member and the second jaw member securely engage the suture to reduce or inhibit movement of the suture.

21. The system of claim 20, wherein in (a) the first and second jaw members are separated by a first distance, wherein in (b) the first and second jaw members are separated by a second distance.

22. The system of claim 21, wherein the first distance is greater than the second distance.

23. The system of claim 21, wherein in (c) the first and second jaw members are (i) placed in contact with each other or (ii) separated by a third distance, wherein the third distance is less than the first distance and the second distance.

24. The system of claim 21, wherein in (b) the suture engagement surfaces of the first and second jaw members are spaced apart to enable the suture to slidably move using the post as a pivot point and bearing surface.

25. The system of claim 15, wherein the suture engagement surfaces of the first and second jaw members comprise one or more serrations.

26. The system of claim 25, wherein the suture engagement surface of the first jaw member comprises a first set of serrations and the suture engagement surface of the second jaw member comprises a second set of serrations complementary to the first set of serrations.

27. The system of claim 15, wherein the pivot post comprises a cylindrical shape.

28. The system of claim 15, further comprising one or more robotic arms, wherein the surgical grasper is coupled to at least one of the one or more robotic arms.

29. The system of claim 28, wherein the one or more robotic arms comprise a first robotic arm comprising a suturing device and a second robotic arm comprising a suture handling device.

30. The system of claim 29, wherein the suturing device comprises a curved needle that is configured to draw a suture through a tissue.

31. The system of claim 29, wherein the suture handling device comprises the surgical grasper.

32. The system of claim 28, further comprising a controller operatively coupled to the one or more robotic arms to control an operation or a movement of the one or more robotic arms to cooperatively perform a suturing procedure on a subject.

33. The system of claim 32, wherein the suturing procedure comprises placing and knotting the suture in a tissue.

34. The system of claim 32, wherein the controller is configured to autonomously control the operation or the movement of the one or more robotic arms.
